(19) United States
(12) Patent Application Publication
(10) Pub. No.: US 2011/0059148 A1
(43) Pub. Date: Mar. 10, 2011

(54) Flexible Drug Delivery Chip, its Fabrication Method and Uses Thereof

(75) Inventors: San-Yuan Chen, Hsinchu City (TW); Wei-Chen Huang, Jhongli City (TW); Dean-Mo Liu, Jhubei City (TW)

(73) Assignee: NATIONAL CHIAO TUNG UNIVERSITY, Hsinchu (TW)

(21) Appl. No.: 12/554,935

(22) Filed: Sep. 7, 2009

(51) Int. Cl. A61F 2/00; A61K 31/4015; A61P 25/08; C25D 13/00

(52) U.S. Cl. 424/423; 514/425; 204/486; 977/773

(57) ABSTRACT

Nanodevice and method for in vivo monitoring and release of drugs are provided. The disclosed nanodevice is characterized in having a drug-loaded nanosphere that is capable of releasing the encapsulated drugs upon magnetically stimulation. The nanodevice may also be used as a contrast agent for in vivo imaging and monitoring the concentration and distribution of the released drugs and/or active compounds injected separately into a target site of a subject.

### BACKGROUND

1. Technical Field

This disclosure is generally in the field of implantable drug delivery devices, and more particularly in the field of devices for the controlled release of a drug from a device implantable in a body lumen or cavity, the method for fabricating such device and uses thereof.

1. Description of Related Art

Medication can be delivered to a patient through a variety of methods, including oral ingestion, inhalation, transdermal diffusion, subcutaneous and intramuscular injection, parenteral administration and implants. Oral drug delivery remains the most preferred way of administration of a medication. However, many current drug delivery products such as oral capsules and tablets possess drawbacks such as limited effectiveness on controlled drug delivery that results in too rapid and incomplete absorption of the drug, irritation of the gastrointestinal tract and other side effects. Further, they may not provide localized therapy, and/or real time monitoring of the distribution of the released drug. Accordingly, a variety of devices and methods have been developed to deliver drug in a more targeted manner.

The microdevices for local drug delivery are developed in response to such need and they may address many of the problems associated with systemic drug delivery. Activation of the drug release may be passively or actively controlled. Examples of controlled drug delivery devices are disclosed in U.S. Pat. No. 6,808,522 and U.S. Pat. No. 6,875,208. Implantable device is particularly useful for the treatment of some maladies that are difficult to treat with currently available therapies and/or require administration of drugs to anatomical regions to which access is difficult to achieve. One example is cancer, where large dose of highly toxic chemotherapeutic agents such as rapamycin, irinotecan (CPT-11) are typically administered to the patient intravenously, which may result in numerous undesired side effects outside the targeted area.

Thus, there is an increasing need of an improved implantable drug delivery system and/or device with improved in vivo adaptability and may deliver medication to targeted area with high efficiently and fewer side effects.

This invention designs, manufactures, and employs a novel implantable drug delivery chip that can be actively and remotely released by proper stimulation at a desired body portion of a subject without inducing any undesired side effects.

### SUMMARY

This disclosure relates to a flexible drug delivery chip, a method for fabricating the same and uses thereof. The flexible drug delivery chip is fabricated by using electrophoretic deposition of drug-loaded magnetic core-shell (i.e., Fe₃O₄@SiO₂) nanoparticles onto an electrically conductive flexible substrate. The flexible drug delivery chip is suitable for in vivo implantation into a body portion of a subject and being magnetically activated to release drug from the magnetic core-shell nanoparticles in a controlled manner in accordance with the strength and/or duration of an external applied magnetic field. The flexible chip offers advantages over conventional drug delivery devices by improving dosing precision, ease of operation, wider versatility of elution pattern and better compliance.

It is therefore a first aspect of this disclosure to provide a drug-containing cell, which is a building block for constructing the flexible drug delivery chip. The drug-containing cell includes a flexible substrate and a drug-containing reservoir. The drug-containing reservoir is formed on the flexible substrate and further includes a plurality of side walls defining a drug-containing volume, wherein at least one side of the drug-containing volume is not sealed by the plurality of side walls; a first layer of drug-containing nanoparticles deposited on the flexible substrate in the drug-containing volume; a layer of metal deposited on the first layer of drug-containing nanoparticles; and a second layer of drug-containing nanoparticles deposited on the metal layer. In one embodiment, the drug-containing cell comprises two metal layers with each metal layer being sandwiched between two layers of drug-containing nanoparticles.

In one example, the plurality of side walls are made of a biocompatible material selected from the group consisting of polyvinylchloride (PVC), polylactide, polyethylene, ethylene-vinyl acetate, polyimides, polyamides, polyethylene glycol, polycaprolactone (PCL), polycolide, polydioxanone, and derivatives and copolymers thereof. The flexible substrate is made of a material selected from the group consisting of polyethylene terephthalate (PET), poly (vinyl chloride) (PVC), polyethylene naphthalate (PEN), polyimide (PI) and polyaryletheretherketone (PEEK). Each of the drug-containing nanoparticles comprises a magnetic iron oxide-containing core and a silicon dioxide shell, and the drug is encapsulated within the magnetic iron oxide-containing core. In one example, the drug is an antieleptic agent. The metal is selected form the group consisting of Au, Ag, Pt, and Ta. In one example, the metal is Au.

In a second aspect of this disclosure, there provides a method of fabricating a drug-containing cell. The method includes steps of: providing a flexible substrate; constructing a drug-containing reservoir by forming a plurality of side walls on the flexible substrate to define a drug-containing volume, wherein at least one side of the drug-containing volume is not sealed by the plurality of side walls; electrophoretically depositing a first layer of drug-containing nanoparticles on the flexible substrate in the drug-containing volume, forming a layer of metal on the first layer of drug-containing nanoparticles by sputter deposition; and electrophoretically depositing a second layer of drug-containing nanoparticles on the metal layer.

In one example, electrophoretic deposition is performed by steps of: (1) providing an electrophoretic deposition cell, which comprises: a colloidal suspension containing about 0.01-30% by weight of drug-containing nanoparticles; and a pair of electrodes; (2) immersing the flexible substrate having constructed thereon the drug-containing reservoir in the colloidal suspension in the electrophoretic deposition cell; and (3) applying a voltage of about 1-50 V to the pair of electrodes for a period of about 1-30 min or until the layer of drug-containing nanoparticles has a thickness of at least 0.1 μm. The colloidal suspension is prepared by suspending the drug-containing nanoparticles in a diluting medium selected from the group consisting of water, a C₁₋₆ alcohol, glycol, glycerin, dimethyl sulfoxide and a combination thereof. Each electrode of the pair of electrodes in the electrophoretic deposition cell is spaced apart from each other for a distance of about 0.5 cm to about 5 cm. The electrophoretic deposition is carried out at a temperature ranges from about −10° C. to about 70° C.

In another example, the sputter deposition is any of ion-beam sputtering, reactive sputtering, ion-assisted deposition, high power impulse magnetron sputtering (HIPIMS) or gas flow sputtering. The metal is selected form the group consisting of Au, Ag, Pt, and Ta. In one example, the metal is Au.

In a third aspect of this disclosure, there provides a flexible drug delivery chip, which is composed of two drug-containing cells of this disclosure arranged in a head-to-head configuration so as to form a drug-releasing chamber. Each of the two drug-containing cells is characterized in having one metal layer sandwiched between two layers of drug-containing nanoparticles. The drug-releasing chamber is characterized in having one side of the chamber being exposed to the surrounding environment thereby providing an outlet for drug elution. In one example, the drug encapsulated within each of the nanoparticles is controlled released from the drug-releasing chamber by applying an external magnetic field with a power from about 0.05 kA/m to 2.5 kA/m for a period of about 10 sec to 180 sec. The flexible drug delivery chip has a thickness of no more than 0.5 mm.

These and other features, aspects, and advantages of the present invention will become better understood with reference to the following description and appended claims.

It is to be understood that both the foregoing general description and the following detailed description directed to the uses and application of such nanodevice are not strictly limited to the ranges described in those examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1(a) is a schematic diagram of a drug-containing cell in accordance with one example of this disclosure;

FIG. 1(b) is the schematic cross-sectional view of the drug-containing cell of FIG. 1(a);

FIG. 2 is a flow chart detailing steps for forming the drug-containing cell of FIG. 1 in accordance with one particular embodiment of this disclosure;

FIG. 3(a) is a schematic diagram illustrating the drug-delivery chip constructed from two drug-containing cells of FIG. 1 in accordance with one particular embodiment of this disclosure;

FIG. 3(b) illustrates the mechanical flexibility exhibited by the drug-delivery chip of FIG. 3(a);

FIG. 4 illustrates high resolution transmission electron microscopy (HRTEM) photographs of Fe₃O₄@SiO₂ nanoparticles prepared in accordance with one embodiment of this disclosure;

FIG. 5 illustrates Fourier Transform Infrared Spectroscopy spectra of Fe₃O₄@SiO₂ nanoparticles, ESM, and ESM-loaded nanoparticles in accordance with one embodiment of this disclosure;

FIG. 6 illustrates drug release profiles for the ESM-loaded nanoparticles in accordance with one embodiment of this disclosure;

FIG. 7 is SEM image at higher magnification reveals a structurally uniform and nonporous morphology of the coating on the substrate in accordance with one embodiment of this disclosure;

FIG. 8 illustrates ESM release profiles of the flexible chip of Example 2 under continuous stimulation of different magnetic field strengths in accordance with one embodiment of this disclosure;

FIG. 9 illustrates drug release profiles of the flexible chip of Example 2 under the various conditions of magnetic induction in accordance with one embodiment of this disclosure;

FIG. 10 illustrate representative examples of spontaneous SWDs under intraperitoneal administration of saline, ethosuximide (ESM) (28 mg/kg, i.p.), ESM with the ESM-loaded nanoparticles (ESM-Fe₃O₄@SiO₂) (40 mg/kg, i.p.), and ESM containing chip (ESM-chip) (ca. 40 mg/kg, intraperitoneal implantation) in accordance with one embodiment of this disclosure; and

FIG. 11 illustrates comparison of SWD number and total SWD duration with saline and 3 different forms of ESM in Long-Evans rats with spontaneous SWDs (n=8), in which (A) illustrates ESM (0.5 ml, 28 mg/kg, i.p.) significantly decreased SWD number and total SWD duration, (B) illustrates ESM loaded nanoparticles (ESM-Fe₃O₄@SiO₂) (40 mg/kg, i.p.) significantly reduced SWD number and total SWD duration, and (C) illustrates ESM-Chip (ca. 40 mg/kg, intraperitoneal implantation) significantly reduced SWD number and total SWD duration. * P<0.01; ** P<0.001.

### DETAIL DESCRIPTION OF THE DISCLOSURE

Reference will now be made in detail to the present embodiments of the invention, examples of which are illustrated in the accompanying drawings.

Described below is a flexible drug delivery chip for in vivo magnetically controlled drug release, its fabrication method and uses thereof. The novel flexible drug delivery chip may be implanted into a desired body portion of a subject and may be actively and remotely controlled to release encapsulated drug to the implant site, such as the arm or any other suitable body portion of a subject, such as a human.

### The Fabrication of a Drug-containing Cell

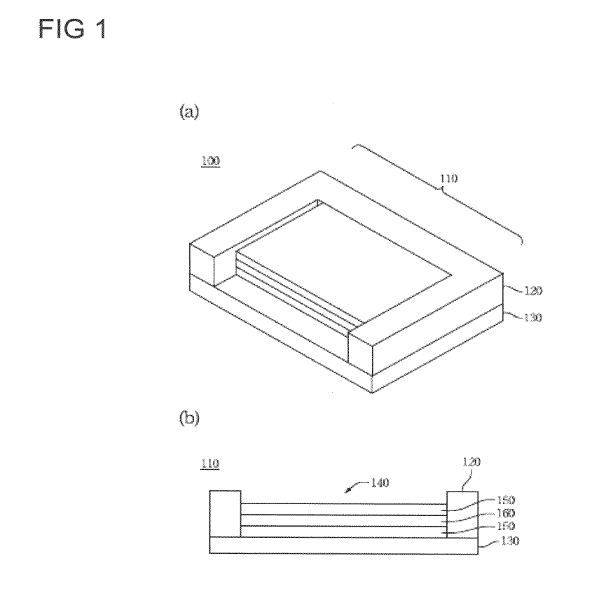

Referring to FIG. 1a, which is a schematic diagram of a drug-containing cell 100. The drug-containing cell 100 is a building block for constructing a flexible drug delivery chip of this disclosure. The drug-containing cell 100 is characterized in having a [symbol] shape drug-containing reservoir 110 defined by a plurality of side walls 120 formed on three sides of a flexible substrate 130 and thereby further defining a drug-containing volume 140 characterized in having at least one side not sealed by the plurality of side walls 120. Referring to FIG. 1(b), which is a front cross-sectional view of the drug-containing reservoir 110 of FIG. 1(a), multiple layers are deposited in sequence within the drug-containing volume 140, including from bottom to top: a first layer of drug-containing nanoparticles 150, a metal layer 160, and a second layer of drug-containing nanoparticles 150.

In an alternative embodiment, the drug-containing reservoir 110 may have two sides, instead of one side, not sealed by the plurality of side walls 120 and thereby is characterized in having an “L” shape or other suitable shape. In the case when the drug-containing reservoir 110 is in “L” shape, two adjacent sides of the reservoir 110 remain exposed to the surrounding environment, thereby forming two outlets for drugs to be released from two directions that are orthogonal to each other.

The flexible substrate for use in this disclosure is generally made of a material selected from the group consisting of polyethylene terephthalate (PET), poly (vinyl chloride) (PVC), polyethylene naphthalate (PEN), polyimide (PI) and polyaryletheretherketone (PEEK). In one example, the flexible substrate 14 is made of PET The plurality of side walls 120 are typically formed from a biocompatible material, which includes, but is not limited to, polyvinylchloride (PVC), polylactide, polyethylene, ethylene-vinyl acetate, polyimides, polyamides, polyethylene glycol, polycaprolactone (PCL), polycolide, polydioxanone, and derivatives and/or copolymers thereof. In one example, the plurality of side walls 16 is made of PVC.

Each of the drug-containing nanoparticles is characterized in having a magnetic iron oxide-containing core and a silicon dioxide shell (e.g., Fe₃O₄@SiO₂), with the drug being encapsulated within the magnetic iron oxide-containing core. The drug-containing Fe₃O₄@SiO₂ nanoparticles may be prepared by a method described previously (Hu et al., J. Nanosci. Nanotechno (2009) 9(2):866-870). In general, each of the nanoparticles has an average diameter of about 10 nm to 100 nm, such as about 10, 11, 12, 13, 14, 15, 16, 17, 18, 19, 20, 30, 40, 50, 60, 70, 80, 90 or 100 nm.

The term “drug” or “biologically active substance” may be used interchangeably herein, and refers to a compound or composition useful for the treatment and/or prevention of conditions in a variety of therapeutic areas and can be administered to a living organism, especially animals such as mammals, particularly humans. The drug useful herein includes, but is not limited to, nucleic acids such as DNA or small interference RNA (siRNA); peptides; proteins such as bovine serum albumin, glycoproteins or collagens; antibiotics; antioxidants such as vitamin E or vitamin C (i.e., ascorbic acid); immunogenic preparations such as a vaccine preparation; an anti-epileptic agent, such as acetazolamide, carbamazepine, clobazam, clonazepam, diazepam, ethosuximide (ESM), ethotoin, felbamate, fosphenytoin, gabapentin, lamotrigine, levetiracetam, mephenytoin, metharbital, methsuximide, methazolamide, oxcarbazepine, phenobarbital, phenytoin, phensuximide, pregabalin, primidone, sodium valproate, stiripentol, tiagabine, topiramate, trimethadione, valproic acid, vigabatrin or zonisamide; an anti-tumor agent such as rapamycin, taxol, camptothecin (CPT), topotecan (TPT) or irinotecan (CPT-11); an anti-bacterial agent such as zinc oxide or quaternary ammonium compounds; an anti-viral agent such as acyclovir, ribavirin, zanamivir, oseltamivir, zidovudine or lamivudine; an anti-proliferative agent such as actinomycin, doxorubicin, daunorubicin, valrubicine, idarubicin, epirubicin, bleomycin, plicamycin or mitomycin; an anti-inflammatory agent such as orticosteroids, ibuprofen, methotrexate, aspirin, salicyclic acid, diphenyhydramine, naproxen, phenylbutazone, indomethacin or ketoprofen; an anti-diabetic agent, which includes sulfonylureas such as tolbutamide, acetohexamide, tolazamide, chlorpropamide, glipizide, glyburide, glimepiride or gliclazide; meglitinides such as repaglinide or nateglinide; biguanides such as metformin, phenformin, or buformin; thiazolidinediones such as rosiglitazone, pioglitazone or troglitazone; alpha-glucosidase inhibitors such as miglitol or acarbose; peptide analogs such as exenatide, liraglutide, taspoglatide, vildagliptin, sitagliptin or pramlintide; and a hormone such as insulin, epidermal growth factor (EGF), and steroids such as progesterone, estrogen, corticosteroids and androgens. In one example, the drug is an antieleptic agent, such as ethosuximide (ESM). Suitable amount of drug that may be encapsulated with the magnetic iron oxide-containing core must be determined empirically. According to one embodiment of this disclosure, the amount of drug in each nanoparticle ranges from about 0.01% to 80% (wt %), such as 0.01, 0.05, 0.1, 0.2, 0.5, 1, 2, 5, 8, 10, 12, 15, 18, 20, 22, 25, 28, 30, 32, 35, 38, 40, 42, 45, 48, 50, 52, 55, 58, 60, 62, 65, 68, 70, 72, 75, 78 or 80%.

The metal layer 170 usually comprises a metal that is selected form the group consisting of Au, Ag, Pt, and Ta. In one example, the metal is Au.

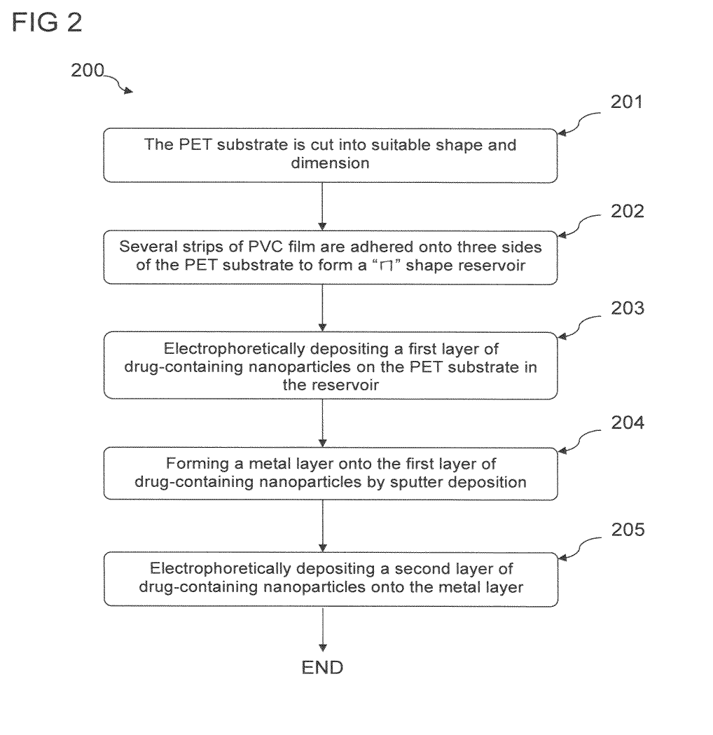

Referring to FIG. 2, which is a flow chart detailing steps for forming the drug-containing cell 100 of FIG. 1 in according to one particular embodiment of this disclosure. The method begins with the step 201, in which a polyethylene terephthalate (PET) substrate is cut into suitable dimension, such as 20 mm×50 mm×0.02 mm. In step 202, several strips of PVC film are adhered onto the PET substrate to form a [symbol] shape reservoir, which is characterized in having one side of the reservoir not sealed by the PVC strips. In steps 203 to 205, multiple layers are deposited in sequence within the reservoir, including a first layer of drug-containing nanoparticles (step 203), a metal layer (step 204) and a second layer of drug-containing nanoparticles (step 205). The layer of drug-containing nanoparticles in step 203 or 205 is formed by electrophoretic deposition (EPD), and the metal layer in step 204 is formed by sputter deposition (SD).

Electrophoretic deposition is typically carried out by steps of: (1) providing an electrophoretic deposition cell, which comprises: a colloidal suspension containing about 0.01-30% by weight of drug-containing nanoparticles; and a pair of electrodes; (2) immersing a flexible substrate (e.g., the PET substrate) having constructed thereon the drug-containing reservoir in the colloidal suspension in the electrophoretic deposition cell; and (3) applying a voltage of about 1-50 V to the pair of electrodes for a period of about 1-30 min or until the layer of drug-containing nanoparticles has a thickness of at least 0.1 μm. The colloidal suspension is prepared by suspending the drug-containing nanoparticles in a diluting medium selected from the group consisting of water, a C₁₋₆ alcohol, glycol, glycerin, dimethyl sulfoxide and a combination thereof. The C₁₋₆ alcohol may be selected from the group consisting of methanol, ethanol, propanol, isopropanol, butanol, isobutanol, sec-butanol, pentanol, isopentanol, hexanol and the like. In one example, the diluting medium is water. In another example, the diluting medium is methanol. The two electrodes in the electrophoretic deposition cell are usually spaced apart from each other for a distance of about 0.5 cm to about 5 cm. In one example, a gap of about 2 cm is formed between the pair of electrodes in the electrophoretic deposition cell. The electrophoretic deposition is carried out at a temperature ranges from about −10° C. to about 70° C.

Sputter deposition is a physical vapor deposition technique for depositing thin film by sputtering, that is, by ejecting material from a source target (e.g., a metal) and then deposited the ejected material onto a substrate (e.g., the PET substrate). Suitable sputter deposition technique for depositing the metal layer in this disclosure may be chosen from any of ion-beam sputtering, reactive sputtering, ion-assisted deposition, high power impulse magnetron sputtering (HIPIMS) or gas flow sputtering. In this example, plasma sputtering is used to sputter deposited a metal layer of Au. The thickness of the metal layer is typically in a range of about 5 to 10 μm. In one example, Au layer has a thickness of about 6.5 μm.

The electrophoretic deposition and the sputter deposition may be respectively repeated for several times, such as 2, 3 or 4 times, depends on the desired volume of the drug-containing reservoir in the drug-containing cell. In one example, the electrophoretic deposition is performed twice, whereas the sputter deposition is performed just once. In other example, the electrophoretic deposition and the sputter deposition may be repeated for the same number of times, such as 2, 3 or 4 times.

### The Construction of a Drug-delivery Chip for In Vivo Magnetically Controlled Drug Release

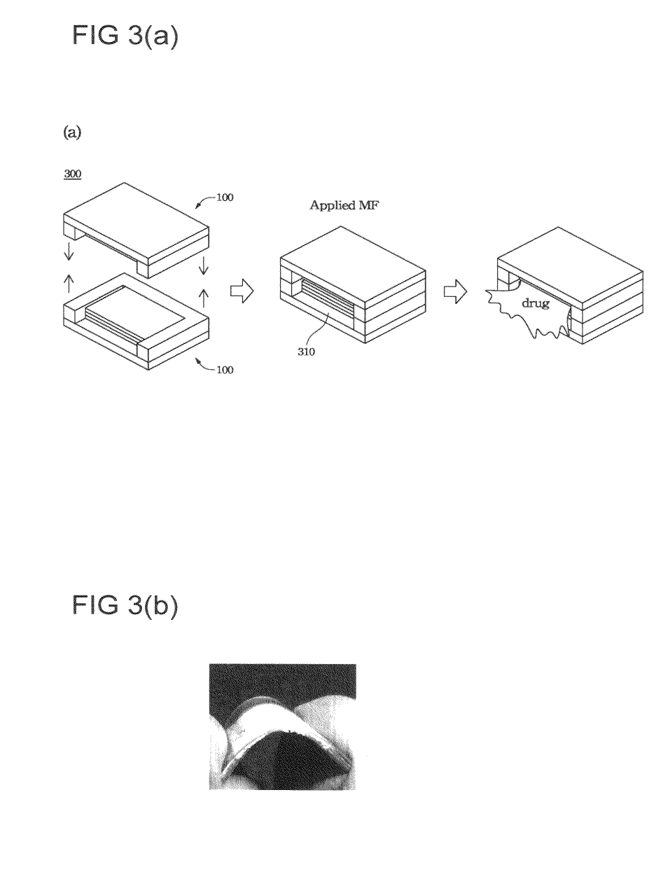

In order to construct a drug-delivery chip 300, two drug-containing cells 100 fabricated in accordance with the steps described above are arranged in a head-to-head configuration, as illustrated in FIG. 3(a). Specifically, one drug-containing cell 100 is inverted and placed on top of the other drug-containing cell 100, so as to join the two drug-containing reservoirs 110 by aligning the plurality of side walls 120 of each reservoir 110 and connecting the two drug-containing volumes 140 and thereby forming a drug-releasing chamber 310. The drug-releasing chamber 310 is characterized in having one side of the chamber being exposed to the surrounding environment and thus provides an outlet for drug elution. The side of the chamber remained exposed to surrounding environment is typically result from connecting the sides of the two drug-containing reservoirs 110 that are not sealed by the plurality of side walls 120. Alternatively, the drug-releasing chamber 310 may provide two outlets, instead of one outlet, for drug elution, if the drug-containing cell 100 having two sides not sealed by the plurality of side walls 120 is used as a building block for constructing the drug-delivery chip 300. The flexible drug delivery chip 300 thus constructed typically has a thickness of no more than 0.5 mm.

The constructed drug-delivery chip 300 may be implanted into a suitable body portion of a subject, such as an arm area, a brain area or the peritoneal lumen or cavity of a human, depends on the disease, condition or disorder that requires medical treatments. The rug-delivery chip 300 of this disclosure also exhibits good mechanical flexibility and therefore is more adaptable to the surrounding environment after implantation. Suitable subject that may benefit from the drug-delivery chip 300 of this disclosure includes, but is not limited to, human or non-human animal. Such non-human animals include all domesticated and feral vertebrates, e.g., mammals, such as primates, dogs, rodents (e.g., mouse or rat), cats, sheep, horses or pigs; and non-mammals, such as birds, amphibians, reptiles and etc. In one example, subjects for whom implantation of the drug-delivery chip 300 may be beneficial include subjects with epilepsy.

The drug encapsulated within each nanoparticle in the layer of drug-containing nanoparticles 150 may be controlled released from the drug-releasing chamber 310 by the application of an external magnetic field (MF) with a power from about 0.05 kA/m to 2.5 kA/m, such as about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 or 2.5 kA/m. The duration of the applied MF may last for a period of about 10 to 180 sec, such as about 10, 20, 30, 40, 50, 60, 70, 80, 90, 100, 110, 120, 130, 140, 150, 160, 170 or 180 sec. Since one side of the drug-releasing chamber 310 remained open to the surrounding environment, hence gradual drug release form the chamber, despite negligible as compared to the magnetically induced drug release, may still be seen in some examples. However, in general, the amount of the drug (e.g., ESM) that is released from the chamber increases as the power of the applied magnetic field increases. In one example, the magnetically induced drug release with a power of 2.5 kA/m is at least 10 folds higher than the amount of gradual drug release. In another example, stepwise drug release profile may be achieved by repeatly turning on and/or off the applied MF at suitable internals. For example, turn the MF on for about 1 min, then shut it off for 10 min, and repeat the on/off action for at least 2, 3, 4 or 5 times, or until the accumulated amount of the released drug has reached a pre-determined level. Therefore, the effective amount of the drugs in the body portion may be controlled by the strength and/or duration of the MF applied. In other words, the drugs encapsulated within the nanoparticles in the delivery chip may be released in a controlled manner by proper adjusting the strength and/or duration of the applied MF on the body portion of the subject.

Reference will now be made in detail to the embodiments of the invention, examples of which are illustrated in the accompanying drawings. Wherever possible, the same reference numbers are used in the drawings and the description to refer to the same or like parts.

### Examples

The following Examples are provided to illustrate certain aspects of the present invention and to aid those of skilled in the art in practicing this invention. These Examples are in no way to be considered to limit the scope of the invention in any manner.

### Example 1

### Preparation of Drug-Loaded Nanoparticles

### 1.1 Preparation of Core-Shell Fe₃O₄@SiO₂ Nanoparticles

Core-shell Fe₃O₄@SiO₂ nanoparticles were synthesized via conventional microemulsion and sol-gel technology as previously described (Hu et al., J. Nanosci. Nanotechnol. (2008) 8:1-5; Santra et al., Adv. Mat. (2005) 17:2165-2169). Briefly, the monodispersed superparamagnetic iron oxide nanoparticles (Fe₃O₄) were synthesized by a high temperature decomposition of Fe(acac)₃. Two key steps were employed in forming monodispersed nanoparticles. First, growing nuclei at 200° C. and then raising the reaction temperature to 300° C. to permit the iron oxide nanoparticles to grow to uniform size. The iron oxide nanoparticles have an average diameter of 5 nm. To design the core-shell structure, a small amount, 0.5 ml, of the Fe₃O₄ suspension was added to 7.7 ml cyclohexane to create the oil phase, while the aqueous phase was composed of 1.6 ml hexanol and 0.34 ml H₂O. Next, these two phases were mixed following the addition of 2 g octyl phenol ethoxylate as the surfactant to form a water-in-oil phase. After adding 2 g of TEOS and aging the mixture for 6 hours, the core-shell Fe₃O₄@SiO₂ nanoparticles was synthesized successfully by microemulsion and sol-gel process. This permitted hydrolysis and condensation reactions to occur, thereby allowing nanoparticles to be formed through gelation. The synthesized nanoparticles were then examined under a transmission electron microscope (TEM, JEM-2100, Japan) and were characterized using electrophoretic light scattering (ELS) for zeta potential determination.

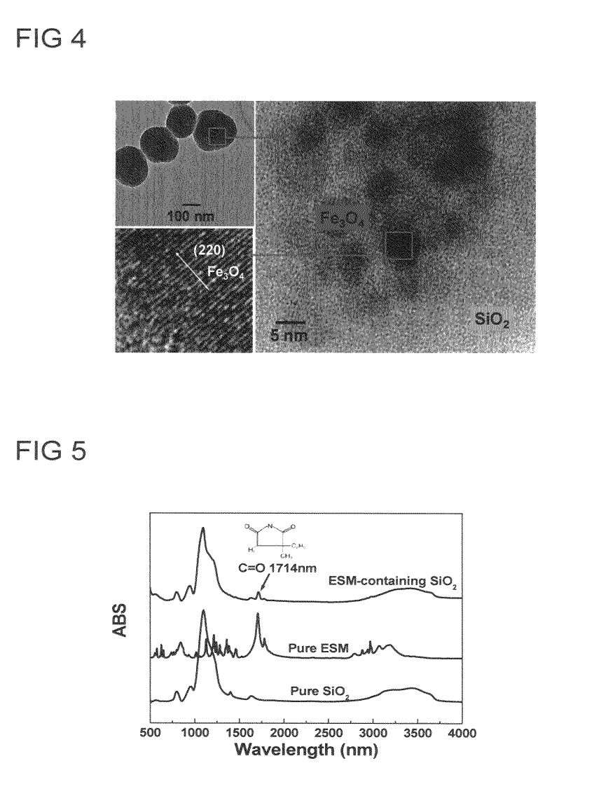

FIGS. 3(a) to 3(c) are high resolution transmission electron microscopy (HRTEM) photographs of the prepared nanoparticles. Results from FIGS. 3(a) to 3(c) confirm that each nanoparticle has a spherical geometry with a mean diameter of about 300 nm, and nanometric magnetic particles were embedded and distributed randomly within the core. Each of the nanoparticles possesses a Fe₃O₄ crystallographic ferrite core and a silica shell. The shell has a thickness of about 5-10 nm. Images presented in FIG. 4 illustrate that the silica shell exhibits a relatively compact structure, where no appreciable pores are detectable under high microscopic resolution.

### 1.2 Drug Encapsulation and Characterization of the Drug-Loaded Nanoparticles

In order to incorporate the hydrophilic anticonvulsant drug, ethosuximide (ESM), into the core-shell nanoparticles, the drug was first dissolved completely in aqueous solution, with a concentration of 5 wt %. The drug was incorporated using an emulsification process described previously for the synthesis of Fe₃O₄@SiO₂ nanoparticles (Hu et al., (2009) supra). After encapsulation of the drug in the core phase of the nanoparticle, a subsequent SiO₂ layer deposition was applied to form a thin outer shell phase, acting as a barrier to regulate the drug release profile.

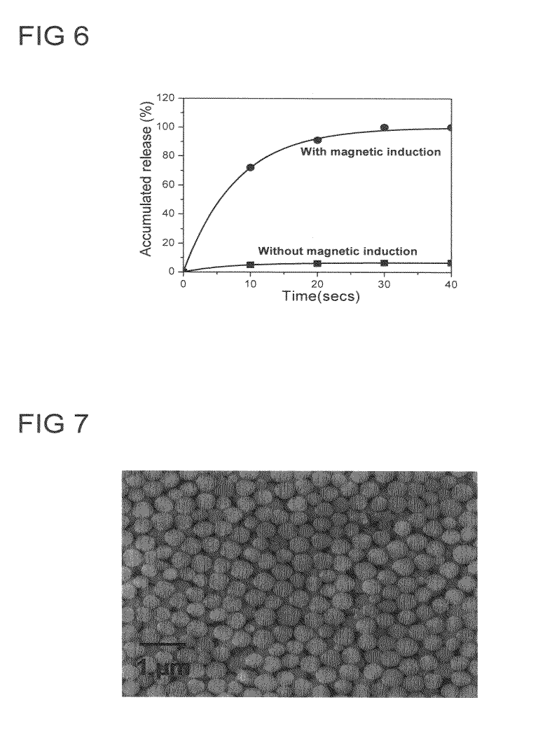

The ESM encapsulated within the Fe₃O₄@SiO₂ nanoparticle was confirmed using Fourier Transform Infrared Spectroscopy (FTIR) analysis, as shown in FIG. 5 where the characteristic peak at the position of 1714 nm⁻¹ designates the C═O bond in ESM for the ESM-containing nanoparticles. The amount of ESM associated with the nanoparticles was determined by applying a magnetic field to allow the complete release of the drug into the environment within a short magnetic induction period, as demonstrated in an earlier study (See Santra et al., (2005) supra). FIG. 6 illustrates the drug release profiles for the Fe₃O₄@SiO₂ nanoparticles with and without magnetic induction. The full strength of the magnetic field is 2.5 kA/m. Under the full magnetic strength of the field, aliquot amounts of the buffer solutions were withdrawn at 10-second intervals, the concentration of ESM released was measured via HPLC, and the cumulative amount of drug release was determined using Equation (1):

$$Cumulative released ( \% ) = \frac{{R}_{t}}{L} \times 100 \% \qquad ( 1 )$$

where L and Rₜ represent the initial amount of drug loaded and the cumulative amount of drug released at time t, respectively.

There is little or no release detectable in the absence of the magnetic field. Although a small amount of drug (about 4-5%), which reaches a relatively constant level after only 20 seconds of immersion, was measured. It is believed that this represents the washing off of surface residue remaining from nanoparticle preparation. Nonetheless, the vast majority of ESM is released (˜100%) with a relatively short induction period (30-40 seconds). This indicates a burst release profile that can be easily managed. This test not only reveals an encapsulation efficiency of about 10%, but the resulting release profiles also suggest the fast-response behavior of the Fe₃O₄@SiO₂ nanoparticles to magnetic induction.

Zeta potential analysis reveals that the isoelectric point (IEP) of the nanoparticles is located in a highly-acidic region, as given in Table 1. As such, the Fe₃O₄@SiO₂ nanoparticles show an increasingly negative charge over a wide pH range of ˜3 to ˜12. In comparison to the zeta potential of pure SiO2 nanoparticles, it appears that the incorporation of Fe₃O₄ could slightly neutralize the negative charging of the silica shell. The highly negative charged Fe₃O₄@SiO₂ nanoparticles, after re-dispersing in ethanol, showed a relatively stable suspension for at least 24 hours.

TABLE 1
Zeta potential analysis reveals the IEP of the nanoparticles located
over a wide range of pH from ~3 to ~12.

| | Zeta potential | Zeta potential of |
|---|---|---|
| | of SiO₂ | SiO₂@Fe₃O₄ |
| pH value | (mV) | (mV) |
| 2.85 | −36 | −11 |
| 5.48 | −51.6 | −41.3 |
| 9.06 | −53.5 | −42.9 |
| 11.82 | −54.2 | −47 |

### Example 2

### Fabrication of a Drug Delivery Chip

### 2.1 Fabrication of a Drug-Containing Cell

To set up an electrophoretic deposition cell, an ITO (In-doped SnO₂)-coated conducting flexible plate (PET substrate, JoinWill Tech.Co., Ltd, Taiwan, having an electrical resistance of sq/50Ω) with dimensions of 20 mm×50 mm×0.02 mm was used as an anode and was patterned by lamination with PVC tape for three side walls of the substrate in advance (See FIG. 1), leaving one side unsealed as the outlet for drug elution. Final lamination of the substrate was performed at a later stage of the assembly. The area of the PET substrate that can be used for membrane deposition is pre-designed to be 1 cm² (1 cm×1 cm). A stainless steel plate (316L) with dimensions of 20 mm×50 mm×0.02 mm (YEONG-SHIN Co., LTD, Taiwan ) was used as cathode and was carefully cleansed sequentially by sonication in acetone, ethanol, and deionized water at room temperature. After the cathode was cleaned and rinsed, it was then dried by blowing with nitrogen gas. A pair of parallel electrodes with 2 cm separation was set vertically in a glass beaker containing the colloidal suspension with 5 wt % of the ESM-containing nanoparticles of Example 1.2 under magnetic agitation. A constant dc voltage (30 V) was applied between two electrodes for 10 minutes. After deposition, the substrate was carefully withdrawn from the glass beaker and dried at room temperature for 1 hour. Under a constant electrical field of 30 V, the ESM-containing nanoparticles of Example 1.2 deposited onto the anodic substrate and reached a thickness of ˜22 um with a deposition time of 10 minutes, corresponding to a deposition rate of about 2.2 um/min. Scanning electron microscopy revealed that a uniform and porous coating can be formed (FIG. 7), indicating that the nanoparticles can be successfully assembled with an orderly-arranged configuration, on the flexible substrate.

After the first deposition of the ESM-containing nanoparticles of Example 1.2 (as the first layer), the flexible substrate was subjected to further coating with a thin layer of sputtered gold to cover the membrane. The purpose of the thin gold coating is twofold; first, it prevents undesired detachment of the deposited nanoparticles from the first layer, and the second, it imparts conductivity for further deposition. Following the same procedure, the second and third layers of the coatings were carried out to form the final drug-containing cell. The second and third layers of the nanoparticle-assembled membranes can be deposited consecutively after a thin coating of Au is applied.

After ambient drying, a structural analysis showed that the resulting multilayer membrane exhibits a thickness of about 70 um and demonstrates excellent structural integrity. Additionally, no considerable delaminated fragments were ever detected. Microscopic examination also indicated a well packed configuration of the drug nanocarriers, leaving nanometric inter-particle voids distributed uniformly throughout the entire membrane. Such a well-packed nanostructure is suggested to stem from strong inter-particle repulsion occurring as a result of the nanoparticles substantial negative charge. The zeta potential reaches a level of about −42 mV at neutral conditions, which should energetically regulate the assembly of the nanoparticles upon impact with the anodic substrate. Despite this, estimates indicate that about 350 nanoparticles reach the substrate over a surface area of 1 um² per minute.

### 2.2 Construction of a Drug Delivery Chip

A drug delivery chip was constructed by use of two drug-containing cells of Example 2.1 arranged in a head-to-head configuration or as the manner illustrated in FIG. 2. This resulted in one side of the chip remaining exposed to the surrounding environment thereby providing an outlet for drug elution. The experimental setup is presented schematically in FIG. 2a. The resulted chip-like device has a total thickness of less than 0.5 mm and is mechanically flexible (FIG. 2b).

### 2.3 Characterization of the Drug Delivery Chip of Example 2.2

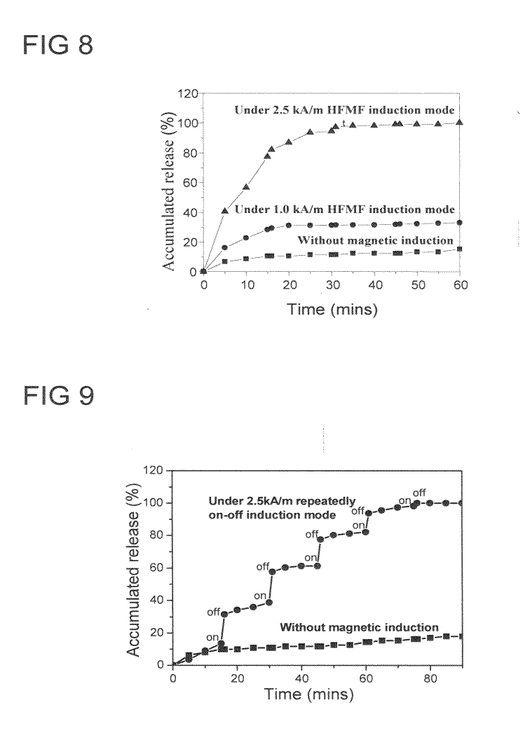

The drug delivery chip of Example 2.2 was subjected to in vitro drug release test. Briefly, a magnetic field was generated, using a home-made AC magnetic generator, with a constant frequency of 70 kHz to trigger drug elution from the flexible drug-carrying chip to examine the release capabilities of said chip. Drug concentration before and after the release tests was assessed by reverse phase high-performance liquid chromatography (RPHPLC) (AGILENT TECHNOLOGIES Co., LTD, Taiwan). Drug solutions were handled at a constant volume of 50 ul, which was injected into the HPLC. The Diode Array Detector was set at 217 nm to detect the anti-epileptic drug, ESM. The mobile phase consisted of 50% water, as phase A, and 50% Methanol, as phase B, with a flow rate of 1.0 mL/min, a gradient elution profile of 5.0-50.0% B with a linear ramp from 0-5 min, 50.0-5.0% B with a linear ramp from 5-10 min, and finally, a 10-min washout period. Drug-free nanoparticle suspensions were prepared as standard controls by immersing Fe₃O₄@SiO₂ particles in water at a concentration of 3% v/v. Test samples were prepared using the same procedure, where 3% v/v Fe₃O₄@SiO₂ encapsulated with ESM was used for drug release tests under various durations of magnetic induction. Results were illustrated in FIG. 8.

FIG. 8 illustrates the release profiles of ESM from the flexible chip under magnetic fields (MF) of varying strengths: 0 A/m (i.e., no MF), 1.0 k A/m, and 2.5 k A/m. The amount of ESM released without magnetic induction was relatively low; about 9-10% over a time span of 60 minutes. In comparison, under a stronger magnetic field, 1.0 kA/m, 40% of ESM was released, and the strongest magnetic field, 2.5 kA/m, produced 100% drug release within the same 60 minute time period. These field-strength specific release profiles strongly indicate that the applied magnetic field effectively drives the release of the drug from the deposited membrane.

A stepwise change in the drug eluting profile can be detected at various time intervals upon repeated on-off magnetic operation, as is seen in FIG. 9. In the presence of magnetic induction, a rapid response in drug elution from the chip is observed with the form of a burst-like profile. With no induction, the drug eluting profile becomes relatively slow. From our understanding, the slower release profile is essentially a consecutive outward diffusion behavior of the drug from inside the chip. This is a direct result of rapid removal from the previous-stage induction, rather than a true release profile of the chip. On this basis, a zero- or near zero-release profile can be reasonably achieved, which provides another alternative drug release mode for the chip.

### Example 3

### In Vivo Drug Release from the Drug Delivery Chip of Example 2

In this example, adult male Long-Evans (N=60) rats were randomly divided into 4 treated groups (n=15 in each group). All rats were kept in a sound-attenuated room under a 12:12 hour light-dark cycle (07:00-19:00 lights on) with food and water provided ad libitum. The experimental procedures were reviewed and approved by the Institutional Animal Care and Use Committee. Briefly, the recording electrodes were implanted under pentobarbital anesthesia (60 mg/kg, i.p.). Subsequently, the rat was placed in a standard stereotaxic apparatus. In total, six stainless steel screws were driven bilaterally into the skull overlying the frontal (A +2.0, L 2.0 with reference to the bregma) and occipital (A −6.0, L 2.0) regions of the cortex to record cortical field potentials. A ground electrode was implanted 2 mm caudal to lambda. Dental cement was applied to fasten the connection socket to the surface of the skull. Following suturing to complete the surgery, animals were given an antibiotic (chlortetracycline) and housed individually in cages for recovery. Long-Evans rats are used because they often display spontaneous SWDs, which have been demonstrated to be associated with absence seizures in several aspects of evidence. In this preliminary animal test, we compared effect among saline, ethosuximide (ESM), ESM-loaded nanoparticles (ESM-Fe₃O₄@SiO₂) and ESM-containing chip (ESM-Chip) in spontaneous SWDs of Long-Evans rats. The chip of Example 2.3 (5 mm×5 mm×0.02 mm) was implanted into the peritoneum of the rats, while the other three doses were subjecting to IP injection.

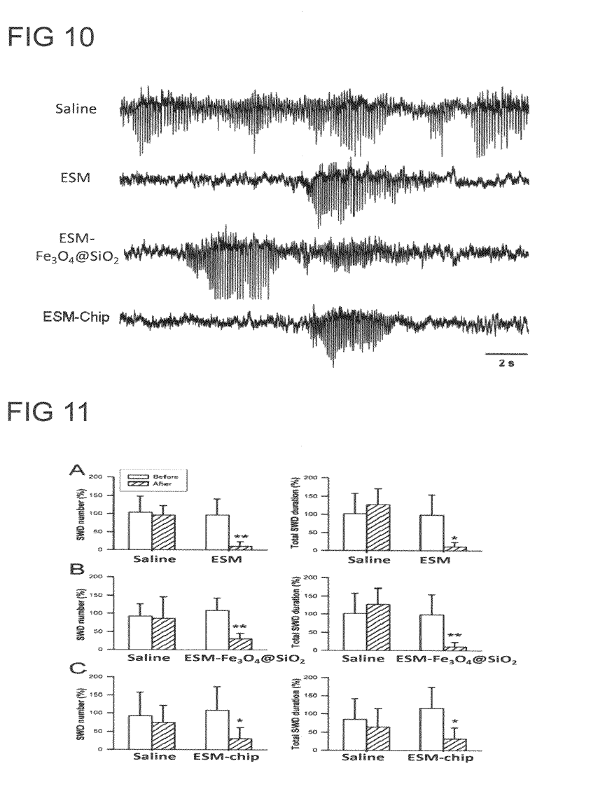

FIG. 10 depicts representative examples of spike-wave discharges (SWDs) after the administration of saline, ESM, ESM-Fe₃O₄@SiO₂, and magnetically-induced ESM-Chip. The SWDs showed no obvious difference. In this experiment, we recorded 1-hour spontaneous brain activity before the treatment (baseline) and another 1-hour spontaneous brain activity 30 minutes after the treatment. The indexes were normalized by average of the two 1-hour baselines. In the conditions of administering ESM-Fe₃O₄@SiO₂ and magnetically-triggered ESM-Chip, rats were restrained in a plastic box then put into the center of a coil following by magnetic stimulus (2.5 kA/m) as the one being aforementioned in vitro to release ESM. Although it is hard to quantify the amount of the ESM released into the rats, results surely indicated that the amount of ESM released, from both ESM-Fe₃O₄@SiO₂ (FIG. 10B) and ESM-Chip (FIG. 10C), demonstrated significant effect in reducing the number and total duration of spontaneous SWDs, as compared to ESM alone (FIG. 10A). Although a peritoneum implantation was employed, instead of brain site, controlled ESM release from these in-vivo data, albeit relatively preliminary, evidenced that the ESM loaded nanoparticles as well as ESM-chip can be successfully eluted through an external magnetic stimulus, as that observed in vitro. In the meantime, the therapeutic efficacy of the ESM being eluted appeared to preserve its effect in SWD suppression.

The foregoing description of various embodiments of the invention has been presented for purpose of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A drug-containing cell, comprising:
a flexible substrate; and
a drug-containing reservoir formed on the flexible substrate comprising:
a plurality of side walls defining a drug-containing volume, wherein at least one side of the drug-containing volume is not sealed by the plurality of side walls;
a first layer of drug-containing nanoparticles deposited on the flexible substrate in the drug-containing volume;
a layer of metal deposited on the first layer of drug-containing nanoparticles; and
a second layer of drug-containing nanoparticles deposited on the layer of metal.

2. The drug-containing cell of claim 1, wherein the plurality of side walls are made of a biocompatible material that is selected from the group consisting of polyvinylchloride (PVC), polylactide, polyethylene, ethylene-vinyl acetate, polyimides, polyamides, polyethylene glycol, polycaprolactone (PCL), polycolide, polydioxanone, and derivatives and copolymers thereof.

3. The drug-containing cell of claim 1, wherein the flexible substrate is made of a material selected from the group consisting of polyethylene terephthalate (PET), poly (vinyl chloride) (PVC), polyethylene naphthalate (PEN), polyimide (PI) and polyaryletheretherketone (PEEK).

4. The drug-containing cell of claim 1, wherein each of the drug-containing nanoparticles comprises a magnetic iron oxide-containing core and a silicon dioxide shell, and a drug is encapsulated within the magnetic iron oxide-containing core.

5. The drug-containing cell of claim 4, wherein the drug is ethosuximide.

6. The drug-containing cell of claim 1, wherein the metal is selected form the group consisting of Au, Ag, Pt, and Ta.

7. The drug-containing cell of claim 1, wherein the drug-containing reservoir comprises two metal layers with each metal layer being sandwiched between two layers of drug-containing nanoparticles in the drug-containing volume.

8. A method of fabricating a drug-containing cell, comprising:
providing a flexible substrate;
constructing a drug-containing reservoir by forming a plurality of side walls on the flexible substrate to define a drug-containing volume thereon, wherein at least one side of the drug-containing volume is not sealed by the plurality of side walls;
electrophoretically depositing a first layer of drug-containing nanoparticles on the flexible substrate in the drug-containing volume,
forming a layer of metal on the first layer of drug-containing nanoparticles by sputter deposition; and
electrophoretically depositing a second layer of drug-containing nanoparticles on the metal layer.

9. The method of claim 8, wherein the electrophoretic deposition is performed by steps of:
providing an electrophoretic deposition cell, which comprises:
a colloidal suspension containing about 0.01-30% by weight of drug-containing nanoparticles; and
a pair of electrodes;
immersing the flexible substrate having constructed thereon the drug-containing reservoir in the colloidal suspension in the electrophoretic deposition cell; and
applying a voltage of about 1-50 V to the pair of electrodes for a period of about 1-30 min or until the layer of drug-containing nanoparticles has a thickness of at least 0.1 μm.

10. The method of claim 9, wherein the colloidal suspension is prepared by suspending the drug-containing nanoparticles in a diluting medium selected from the group consisting of water, a C₁₋₆ alcohol, glycol, glycerin, dimethyl sulfoxide and a combination thereof.

11. The method of claim 8, wherein a gap of about 0.5 cm to about 5 cm is formed between the pair of electrodes.

12. The method of claim 8, wherein the electrophoretic deposition is carried out at a temperature ranges from about −10° C. to about 70° C.

13. The method of claim 8, wherein the metal is selected form the group consisting of Au, Ag, Pt, and Ta.

14. The method of claim 8, wherein the plurality of side walls are made of a biocompatible material that is selected from the group consisting of collagen, polyvinylchloride (PVC), polylactide, polyethylene glycol, polycaprolactone (PCL), polycolide, polydioxanone, and derivatives and copolymers thereof.

15. The method of claim 8, wherein the flexible substrate is made of a material selected from the group consisting of polyethylene terephthalate (PET), polyethylene, ethylene-vinyl acetate, polyimides, polyamides, polyethylene naphthalate (PEN), polyimide (PI) and polyaryletheretherketone (PEEK).

16. The method of claim 8, wherein each of the drug-containing nanoparticles comprises a magnetic iron oxide-containing core and a silicon dioxide shell, and a drug is encapsulated within the magnetic iron oxide-containing core.

17. The method of claim 16, wherein the drug is ethosuximide.

18. A flexible drug delivery chip, comprising
two drug-containing cells of claim 1 arranged in a head-to-head configuration so that the drug-containing reservoir of each cell cooperatively forms a drug-releasing chamber with one side of the drug-containing volume not sealed by the plurality of side walls and thereby serves as the side for magnetically induced drug release.

19. The flexible drug deliver chip of claim 18, wherein the drug encapsulated within each of the nanoparticles is controlled released from the drug-releasing chamber by the application of a magnetic field with a power from about 0.05 kA/m to 2.5 kA/m for a period of about 10 sec to 180 sec.

20. The flexible drug deliver chip of claim 19, wherein the drug is ethosuximide and the chip has a thickness of no more than 0.5 mm.
